(19) United States
(12) Patent Application Publication
(10) Pub. No.: US 2003/0162754 A1
(43) Pub. Date: Aug. 28, 2003

(54) Use of GABA and GABAB agonists

(75) Inventor: Brooke Ligon, Franklin, MA (US)

Correspondence Address: NIXON PEABODY LLP, 101 FEDERAL ST, BOSTON, MA, 02110, US

(73) Assignee: Tufts University

(21) Appl. No.: 10/321,709

(22) Filed: Dec. 17, 2002

Related U.S. Application Data
(60) Provisional application No. 60/341,520, filed on Dec. 17, 2001.

(51) Int. Cl. A61K 31/66; A61K 31/4172; A61K 31/381; A61K 31/198

(52) U.S. Cl. 514/114; 514/557; 514/561; 514/447; 514/565; 514/400

(57) ABSTRACT

The present invention provides methods of stimulating tissue growth, including islet cell growth, by administering GABA or a GABA agonist to act on GABA_{B} receptors and GABA_{B}-like receptors to activate cell replication.

This application claims the benefit of U.S. Provisional Application No. 60/341,520, filed Dec. 17, 2001.

### FIELD OF THE INVENTION

The present invention relates to the use of GABA and GABA_{B} receptor agonists to stimulate tissue growth, including pancreatic islet replication and neogenesis.

### BACKGROUND OF THE INVENTION

GABA (γ-aminobutyric acid) is an endogenous neurotransmitter in the central and peripheral nervous systems. Receptors for GABA have traditionally been divided into GABA_{A} and GABA_{B} receptor subtypes. GABA_{B} receptors (for a review see Kerr, D. I. B. and Ong, J. (1995) Pharmac. Ther. vol. 67, pp. 187-246) belong to the superfamily of G-protein coupled receptors.

In the CNS, GABA is known to exert its actions through at least two distinct receptor types-ionotropic GABA_{A} receptors (which form Cl⁻ channels) and metabotropic GABA_{B} receptors (members of the C family of G protein-coupled receptors). The recent cloning and heterologous expression of GABA_{B} receptor cDNAs has revealed the heterodimerization of two distinct G-protein coupled receptors, GBR1 and GBR2, that appears to be required to activate some downstream effectors e.g. G protein activated K⁺ channels, and thus provides additional means to investigate ways in which GABA is employed peripherally (Kaupmann et al., Nature 386(6622), 239-246, Mar. 20, 1997). In addition, the carboxy terminal coiled coil domain involved in subunit dimerization has also been shown to bind transcription factors. White, J. H., McIllhinney R. A. J., Wise, A., Ciruela, F., Chan, W-Y., Emson, P., C., Billinton, A., Marshall, F. H. The GABA_{B} receptor interacts directly with the related transcription factors CREB2 and ATFx. Proc. Natl. Acad. Sci 97 13967-13972 (2000). Although GABA and the synthesis enzyme, glutamic acid decarboxylase (GAD), are also expressed in peripheral organs, the physiologic role of the transmitter outside the CNS is less clear.

GABA_{B} receptor agonists are described as being of use in the treatment of CNS disorders, such as muscle relaxation in spinal spasticity, cardiovascular disorders, asthma, gut motility disorders such as irritable bowel syndrome and as prokinetic and anti-tussive agents. GABA_{B} receptor agonists have also been disclosed as useful in the treatment of emesis (U.S. Pat. No. 5,719,185).

The GABA_{B} receptor agonist baclofen (4-amino-3-(4-chlorophenyl)butanoic acid) (Swiss patent No. CH 449,046) has been the most studied of the GABA analogs. Other GABA_{B} receptor agonists or partial agonists are disclosed in: EP 0356128; EP 0181833; EP 0399949; EP 0463969; and FR 2,722,192. For a review on the chemistry of GABA_{B} modulators, see Forest, W. and Mickel, S. J. in: The GABA Receptors, pp. 271-296 (Eds. S. J. Enna and N. G. Bowery, Humana Press Inc., Totowa, N.J., U.S.A. 1997).

Most peripheral tissues containing GABA express about 1% of GABA expressed in the brain. However, one location of high concentrations of GABA outside of the CNS is in the beta cells of the pancreatic islet. The abundance of GABA in beta cells has been demonstrated to be similar to GABAergic regions of the brain. Additionally, GABA has been shown to be secreted from beta cells in response to glucose and require glutamine availability for further GABA synthesis. One role of this secreted GABA is to regulate glucagon release from alpha cells in islets, producing inhibition through GABA_{A} receptor Cl⁻ channels.

Beta cells in pancreatic islets secrete insulin. When insulin secretion is impaired for any reason, diabetes ensues. There are two primary types of diabetes: insulin-dependent and non-insulin dependent diabetes mellitus (IDDM and NIDDM, respectively). The two forms of the disease are distinguished by a number of features, although both are characterized by varying degrees of insulin deficiency.

In IDDM there is profound insulin deficiency such that even the low levels of insulin which would normally prevent lipolysis and cytogenesis cannot be sustained. IDDM patients generally show high levels of glucose and low levels of insulin. Without replacement of insulin, IDDM patients become ketotic and die. Current therapy, therefore, includes daily insulin injections.

NIDDM is a common and complex disorder which results from a combination of defects in insulin secretion and impaired insulin sensitivity in peripheral tissues. NIDDM is characterized by hyperglycemia in both the fasted and fed states, variable degrees of hyperinsulinaemia and obesity. Current therapies include diet, sulfonylurea to enhance insulin secretion, insulin itself, and biguanides to reduce insulin resistance.

Today, there is no cure for either IDDM or NIDDM. Moreover, while administration of insulin provides significant benefits to patients suffering from insulin-dependent diabetes, the short serum half-life of insulin creates difficulties for maintaining proper dosage. The use of insulin also can result in a variety of hypoglycemic side-effects and the generation of neutralizing antibodies. Similarly, the pills available for non-insulin dependent diabetics do not provide ideal glycemic control and involve drastic lifestyle alterations. In view of the problems associated with existing treatments of diabetes, there is a compelling need for improved treatment and preferably a cure for diabetes.

### SUMMARY OF THE INVENTION

The present invention provides methods of stimulating tissue growth, including islet cell growth, by administering GABA or a GABA agonist to act on GABA_{B} receptors and GABA_{B}-like receptors to activate cell replication.

One preferred embodiment of the invention provides a method for stimulating tissue growth by administering an effective amount of GABA, a GABA_{B} receptor agonist, or a GABA_{B}-like receptor agonist.

Preferably, the administered compound is 4-aminobutanoic acid (GABA), 4amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4amino-3-(thien-2-yl)butanoic acid, 4-amino-3-(5-chlorothien-2-yl)butanoic acid, 4-amino-3(5-bromothien-2-yl)butanoic acid, 4-amino-3-(5-methylthien-2-yl)butanoic acid, 4-amino-3(2-imidazolyl)butanoic acid, 4-guanidino-3-(4-chlorophenyl)butanoic acid, (3aminopropyl)phosphonous acid, (4-aminobut-2-yl)phosphonous acid, sodium butyrate, (3amino-2-methylpropyl)phosphonous acid, (3-aminobutyl)phosphonous acid, (3-amino-2-(4chlorophenyl)propyl)phosphonous acid, (3-amino-2-(4-chlorophenyl)-2hydroxypropyl)phosphonous acid, (3-amino-2-(4-fluorophenyl)propyl)phosphonous acid, (3amino-2-phenylpropyl)phosphonous acid, (3-amino-2-hydroxypropyl)phosphonous acid, (E)(3-aminopropen-1-yl)phosphonous acid, (3-amino-2-cyclohexylpropyl)phosphonous acid, (3amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4methoxyphenyl)propyl]phosphonous acid, [3-amino-2-(4-chlorophenyl)-2hydroxypropyl]phosphonous acid, (3-aminopropyl)methylphosphinic acid, (3-amino-2hydroxypropyl)methylphosphinic acid, (3-aminopropyl)(difluoromethyl)phosphinic acid, (4aminobut-2-yl)methylphosphinic acid, (3-amino-1-hydroxypropyl)methylphosphinic acid, (3amino-2-hydroxypropyl)(difluoromethyl)phosphinic acid, (E)-(3-aminopropen-1 yl)methylphosphinic acid, (3-amino-2-oxo-propyl)methylphosphinic acid, (3aminopropyl)hydroxymethylphosphinic acid, (5-aminopent-3-yl)methylphosphinic acid, (4amino-1,1,1-trifluorobut-2-yl)methylphosphinic acid, (3-amino-2-(4chlorophenyl)propyl)sulfinic acid, and 3-aminopropylsulfinic acid. Baclofen is a preferred compound.

In another preferred embodiment, the invention provides a method a regenerating tissue in a host in need of tissue regeneration, by administering a GABA_{B} receptor agonist or GABA_{B}-like receptor agonist, which can be any one of the compounds listed supra, and preferably is baclofen.

Another embodiment of the invention provides methods for stimulating islet cell replication and islet neogenesis, which are useful in the treatment of insulin-dependent and non-insulin-dependent diabetes mellitus.

One preferred embodiment provides a method of treating a patient with diabetes mellitus by administering GABA and GABA_{B} receptor agonists to control and stimulate growth of insulin-producing islet cells to effectively treat diabetes, while further providing other related advantages. The method of the invention comprises administering to an individual in need of such treatment an effective amount of GABA or a GABA_{B} receptor agonist or a GABA_{B}-like receptor agonist, or a pharmaceutically acceptable thereof. The GABA_{B} receptor agonist or GABA_{B}-like receptor agonist can be any one of the compounds listed supra, and preferably is baclofen.

In one embodiment, the present invention can be used for the treatment of insulin dependent diabetes mellitus. In another embodiment, the invention can be used for the treatment of non-insulin dependent diabetes mellitus.

In yet another embodiment, the method of the present invention can be used for stimulating islet growth ex vivo. The stimulation of the islet growth ex vivo comprises administering to a culture of islet cells an effective amount of GABA, a GABA_{B} receptor agonist, or a GABA_{B}-like receptor agonist, or a pharmaceutically acceptable saltthereof. The cultured islet cells can be transplanted into a patient in need thereof.

It is to be understood that while the invention has been described in conjunction with the preferred specific embodiments thereof that the foregoing description as well as the examples that follow are intended to illustrate and not limit the scope of the invention. Other aspects, advantages and modifications within the scope of the invention will be apparent to those skilled in the art to which the invention pertains.

### BRIEF DESCRIPTION OF THE DRAWINGS

The drawings, which are incorporated in and constitute a part of this specification, illustrate embodiments of the invention and, together with the description, serve to explain the objects, advantages, and principles of the invention. In the drawings:

FIGS. 1A-B show GABA_{B} receptor expression in islets. FIG. 1A is a Northern blot of mRNA purified from rat islets (I), cortex (Ctx), cerebellum (Cer), and spleen (S), probed with a riboprobe complementary to nucleotides *-* of the coding region of GBR1. FIG. 1B is an immunoblot of protein isolated from large (>150 μm diameter) or small (<150 μm diameter) islets as marked, and reacted with the C-terminal antibody to GBR1.

FIGS. 2A-L show GABA_{B} receptor subunits expressed in β cells. Antibodies specific for GBR1 C terminus (FIGS. 2B, D, F, and J), GBR1 N terminus (FIG. 2G), GBR2 (FIG. 2J), GABA (FIG. 2A), and insulin (FIGS. 2C and E) were used to label islets in intact pancreatic tissue (FIGS. 2A, B, G-L) or enzymatically-isolated islets (FIGS. 2C-F). Panels I and L are overlays of the two related images. Scale bars—10 μm.

FIGS. 3A-D show developing islets and ductal cells expressing GBR1 and GABA. Pancreatic tissue was co-immunolabeled with anti-GABA (FIGS. 3A and C) and anti-GBR1 N terminus (FIGS. 3B and D). Arrowheads in panel a highlight ducts. Scale bars =25 μm.

FIGS. 4A-D illustrate that GABA stimulates islet cell growth. FIG. 4A is a histogram of islet DNA content (measured with fluorescence method) after exposure to 100 μM GABA or baclofen in the presence or absence of 100 μm. 20H-saclofen (sac) or bicuculline (bic) or the N terminal antibody to GBR1 (1:1000), as marked. Bars represent means±SEMs for between 4 and 13 independent measurements each. FIGS. 4B-D are micrographs of islets labeled with DAPI (b and d, left panels, c, bottom panel, or BrdU (B, right; D, center; C, top). Right panel in FIG. 4D is an overlay of the two other panels. Scale bars =25 μm.

FIG. 5 is a histogram showing the change in islet DNA content induced by exposure to 100 μM GABA in the presence of glucose concentrations shown on abscissa. Moderate glucose levels are optimal for GABA-induced proliferation. Bars are means±SEMs (n=*).

FIG. 6 is a histogram showing the change in DNA content induced in spleen by exposure to GABA or baclofen.

FIG. 7 is an immunoblot showing expression of proteins which cross-react with an anti-GABA_{B} receptor antibody in protein lysates isolated from cortex, pancreas, islets, and kidney tissue.

FIG. 8 is an immunoblot showing expression of proteins which cross-react with an anti-GABA_{B} receptor antibody in protein lysates isolated from cerebellum, pancreas, islets, and spleen tissue.

### DETAILED DESCRIPTION OF THE INVENTION

We have now surprisingly discovered that GABA can influence tissue growth, including islet growth, when the tissue is treated with GABA, a GABA_{B} receptor agonist, or GABA_{B}-like receptor agonist. More particularly, we have discovered that GABA, by activating the GABA_{B} receptor, serves as an endogenous growth factor for islets as well as other tissues. We have found that multiple isoforms of the GABA_{B} receptor subunits, GBR1 and GBR2, are expressed in the islets of Langerhans. GABA, like insulin, is secreted in response to nutrients and is co-expressed in islets with the GABA_{B} receptor which binds transcription factors. Treatment of islets with GABA or a GABA_{B} receptor agonist, such as baclofen, produces an increase of DNA synthesis. Additionally, the GABA_{B} receptor antagonist, 2OH-saclofen, and an antibody to the GABA_{B} R1 N-terminus inhibit the mitogen effect of GABA on islets. We have further discovered that the GABA agonist baclofen stimulates replication in a non-islet tissue, spleen, and that novel GABA_{B}-like receptors are expressed in tissues including the central nervous system, pancreatic islets, kidney, and spleen. Thus, the present invention provides the use of GABA, GABA_{B} receptor agonists, and GABA_{B}-like receptor agonists for the stimulation of growth of tissues, including insulin-producing islets. and treatment of diseases associated with abnormal insulin-secretion, such as diabetes mellitus.

For the purpose of this invention, the term “agonist” should be understood as including both full agonists as well as partial agonists, whereby a “partial agonist” should be understood as a compound capable of partially, but not fully, activating the GABA_{B} receptor.

Examples of compounds having agonistic or partially agonistic affinity to GABA_{B} receptors and which thus can be used according to the invention are:

4-aminobutanoic acid (GABA),

4-amino-3-(4-chlorophenyl)butanoic acid (baclofen),

4-amino-3-phenylbutanoic acid,

4-amino-3-hydroxybutanoic acid,

4-amino-3-(4-chlorophenyl)-3-hydroxyphenylbutanoic acid,

4-amino-3-(thien-2-yl)butanoic acid,

4-amino-3-(5-chlorothien-2-yl)butanoic acid,

4-amino-3-(5-bromothien-2-yl)butanoic acid,

4-amino-3-(5-methylthien-2-yl)butanoic acid,

4-amino-3-(2-imidazolyl)butanoic acid,

4-guanidino-3-(4-chlorophenyl)butanoic acid,

3-amino-2-(4-chlorophenyl)- 1-nitropropane,

(3-aminopropyl)phosphonous acid,

(4-aminobut-2-yl)phosphonous acid,

(3-amino-2-methylpropyl)phosphonous acid,

(3-aminobutyl)phosphonous acid,

(3-amino-2-(4-chlorophenyl)propyl)phosphonous acid,

(3-amino-2-(4-chlorophenyl)-2-hydroxypropyl)phosphonous acid,

(3-amino-2-(4-fluorophenyl)propyl)phosphonous acid,

(3-amino-2-phenylpropyl)phosphonous acid,

(3-amino-2-hydroxypropyl)phosphonous acid,

(E)-(3-aminopropen-1-yl)phosphonous acid,

(3-amino-2-cyclohexylpropyl)phosphonous acid,

(3-amino-2-benzylpropyl)phosphonous acid,

[3-arnino-2-(4-methylphenyl)propyl]phosphonous acid,

[3-amino-2-(4-trifluoromethylphenyl)propyl]phosphonous acid,

[3-amino-2-(4-methoxyphenyl)propyl]phosphonous acid,

[3-amino-2-(4-chlorophenyl)-2-hydroxypropyl]phosphonous acid,

(3-aminopropyl)methylphosphinic acid,

(3-amino-2-hydroxypropyl)methylphosphinic acid,

(3-aminopropyl)(difluoromethyl)phosphinic acid,

(4-aminobut-2-yl)methylphosphinic acid,

(3-amino-1-hydroxypropyl)methylphosphinic acid,

(3-amino-2-hydroxypropyl)(difluoromethyl)phosphinic acid,

(E)-(3-aminopropen-1-yl)methylphosphinic acid,

(3-amino-2-oxo-propyl)methyl phosphinic acid,

(3-aminopropyl)hydroxymethylphosphinic acid,

(5-aminopent-3-yl)methylphosphinic acid,

(4-amino-1,1,1-trifluorobut-2-yl)methylphosphinic acid,

(3-amino-2-(4-chlorophenyl)propyl)sulfinic acid,

3-aminopropylsulfinic acid.

Preferably, the compound having agonistic or partially agonistic affinity to a GABA_{B} receptor is any one of the following compounds:

4-amino-3-(4-chlorophenyl)butanoic acid (baclofen),

(3-aminopropyl)methylphosphinic acid,

(3-amino-2-hydroxypropyl)methylphosphinic acid,

4-aminobutanoic acid (GABA),

(3-amino-2-(4-chlorophenyl)propyl)sulfinic acid,

(3-aminopropyl)(difluoromethyl)phosphinic acid,

(3-amino-2-oxo-propyl)methyl phosphinic acid,

4-amino-3-(5-chlorothien-2-yl)butanoic acid,

(3-aminopropyl)phosphonous acid.

Methods for synthesizing the above compounds are disclosed supra and in GB 1017439, e.g. baclofen, U.S. Pat. No. 4,656,298, e.g. 3-aminopropylphosphonous acid (3aminopropylphosphinic acid), EP 0356128, i.e. 3-(aminopropyl)methyl phosphinic acid, and EP0463969, e.g. 3-(2-imidazolyl)-4-aminobutanoic acid, which disclosures are incorporated herein by reference.

The use of pharmaceutically acceptable salts of GABA_{B} agonists for the disclosed purposes is also included in the invention. Most known GABA_{B} agonists such as for example baclofen, (3-aminopropyl) methylphosphinic acid and (3-amino-2-(S)hydroxypropyl)-methylphosphinic acid are of amphoteric nature and may be present in the form of internal salts. They also can form acid addition salts and salts with bases. Such salts are particularly pharmaceutically acceptable acid addition salts, as well as pharmaceutically acceptable salts formed with bases. Suitable acids for the formation of such salts include, for example, mineral acids such as hydrochloric, hydrobromic, sulfuric or phosphoric acid or organic acids such as organic sulfonic acids and organic carboxylic acids. Salts of GABA_{B} agonists with bases are, for example, alkali metal salts, e.g., sodium or potassium salts, or alkaline earth metal salts, e.g. calcium or magnesium salts as well as ammonium salts, such as those with ammonia or organic amines.

The use of optical isomers of GABA_{B} agonists for the disclosed purposes is also included in the invention. Many known GABA_{B} agonists such as for example baclofen and (3-amino-2-(S)-hydroxypropyl)methylphosphinic acid are chiral compounds due to the presence of an asymmetric carbon atom. Depending on the presence of asymmetric atoms, the GABA_{B} agonists may be in the form of mixtures of isomers, particularly racemates, or in the form of pure isomers, especially enantiomers.

We have now discovered that GABA and GABA_{B} agonists promote cell growth in tissues that express GABA_{B} receptors and/or GABA_{B}-like receptors.

GABA_{B} receptors and/or GABA_{B}-like receptors are expressed in a wide variety of tissues, including but not limited to the central nervous system, including for example the amygdala, caudate nucleus, cerebellum, corpus collosum, hippocampus, and putamen, as well as peripheral tissues such as lung, pancreas, spleen, small intestine, stomach, prostate, and uterus tissues. Similarly, GABA_{B}-like receptors are expressed in a wide variety of tissues including but not limited to the central nervous system, including the cortex and cerebellum, pancreas, including pancreatic islets, spleen, and kidney. These tissues may be stimulated or regenerated in one embodiment of the present invention. Patient in need of stimulation or regeneration are selected for administration.

GABA_{B}-like receptors are characterized as GABA_{B}-like receptors based on the following features. First, an antibody raised to a peptide in the ligand binding domain of the GABA_{B} receptor consisting of the following residues: RRDILPDYELKLIHHD (SEQ ID NO: 1) recognizes known GABA_{B} receptors as well as several GABA_{B}-like receptors which have different amino acid sequences. Second, these GABA_{B}-like receptors contain portions of the amino acid sequence comprising the peptide used to generate this antibody. Third, the GABA_{B}-like receptors respond to agents which react to GABA_{B} receptors, including baclofen. Fourth, genes encoding these GABA_{B}-like receptors can be cloned from libraries representing rat, mouse, or human genes expressed in the brain, and are 103-130 kilodaltons.

A feature of insulin-dependent diabetes mellitus (IDDM) and at times, noninsulin-dependent diabetes mellitus (NIDDM) diabetes, is the inability of the islet cell mass to increase sufficiently to cope with the demands of sustained high glucose. This is especially apparent in IDDM where there is also profound destruction of existing beta cells. In IDDM a primary focus of the autoimmune attack is GAD, the enzyme required to synthesize the proposed mitogen GABA. Although not wishing to be bound by theory, our data demonstrating the co-localization of GABA and the GABA_{B} receptor, suggests that the cells capable of providing for neogenesis may be targeted for destruction early in IDDM.

The present invention provides a method for the treatment of diabetes mellitus which comprises administration to a mammal, including man, in need of such treatment an effective amount of a GABA_{B} receptor agonist as defined above.

For use as a regulator/stimulator of islet cell growth and the treatment of diabetes, the GABA_{B} receptor agonist may be used at doses appropriate for other conditions for which GABA_{B} receptor agonists are known to be useful. The typical daily dose of the active substance varies within a wide range and will depend on various factors such as for example the individual requirement of each patient and the route of administration. In general, dosages will be in the range of 1 μg to 100 mg per day and kg body weight, preferably 10 μg to 10 mg per day and kg body weight.

For clinical use, the compounds of the invention are formulated into pharmaceutical formulations for oral, rectal, parenteral or other mode of administration. The pharmaceutical formulation contains a compound of the invention in combination with one or more pharmaceutically acceptable ingredients. The carrier may be in the form of a solid, semi-solid or liquid diluent, or a capsule. These pharmaceutical preparations are a further object of the invention. Usually the amount of active compounds is between 0. 1-95% by weight of the preparation, preferably between 0.2-20% by weight in preparations for parenteral use and preferably between 1 and 50% by weight in preparations for oral administration.

In the preparation of pharmaceutical formulations containing a compound of the present invention in the form of dosage units for oral administration the compound selected may be mixed with solid, powdered ingredients, such as lactose, saccharose, sorbitol, mannitol, starch, arnylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatin capsules may be prepared with capsules containing a mixture of the active compound or compounds of the invention. vegetable oil, fat, or other suitable vehicle for soft gelatin capsules. Hard gelatin capsules may contain granules of the active compound. Hard gelatin capsules may also contain the active compound in combination with solid powdered ingredients such as lactose, saccharose, sorbitol, mannitol, potato starch, corn starch, arnylopectin, cellulose derivatives or gelatin.

Dosage units for rectal administration may be prepared (i) in the form of suppositories which contain the active substance mixed with a neutral fat base; (ii) in the form of a gelatin rectal capsule which contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions containing from 0.2% to 20% by weight of the active ingredient and the remainder consisting of sugar or sugar alcohols and a mixture of ethanol, water, glycerol, propylene glycol and polyethylene glycol. If desired, such liquid preparations may contain coloring agents, flavoring agents, saccharin and carboxymethyl cellulose or other thickening agents. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent, preferably in a concentration from 0.1% to 10% by weight. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

Further included in the invention is the use of cells, transfected with a nucleotide sequence encoding a GABA_{B} receptor or GABA_{B}-like receptor, for screening purposes, in order to identify regulators/stimulators of insulin-producing islet cell production. The GABA_{B} receptor or GABA_{B}-like receptor may be any one of the known GABA_{B} receptor subtype genes, such as GABA_{B} RIA or the GABA_{B} R1B or any hitherto uncloned subtypes of the GABA_{B} receptor or GABA_{B}-like receptor. The nucleotide sequences may be derived from any species, but preferably from a mammal and most preferably from man.

The invention will be further characterized by the following examples which are intended to be exemplary of the invention.

### EXAMPLES

GABA_{B} Receptor mRNAs Expressed in Islets

Using primers designed from the sequence of GBR1 isolated from rat brain, reverse-transcription, polymerase chain reaction was used to amplify GBR1 receptor transcripts from polyA+-mRNA purified from islet tissue (FIG. 1A). Several variants were identified: a full-length GBR1a identical to that cloned from rat brain²¹, a variant lacking exon 6 (a portion of the agonist binding pocket²⁰), and three variants similar to GBR1e that code for a long, extracellular ligand binding N-terminal but lack membrane spanning domains²⁴. One of the truncated islet variants contains a 63 base insert that is unique to islets.

To further establish the presence of GABA_{B} receptor transcripts in islets, a *bp region from the 5′ end of GBR1 cDNA was labeled and used as a probe in Northern blot analysis. This probe hybridized to an islet niRNA approximately 5 kb in size, similar to that of the full-length GBRla transcript found in rat cortex and cerebellum (FIG. 1B). Interestingly, this probe also hybridized to an mRNA of similar size in spleen, suggesting that islets are not the only peripheral tissue expressing the GABA_{B} receptor mRNA^{20,24}.

Localization of GABA and GABA_{B} Receptor in Islets

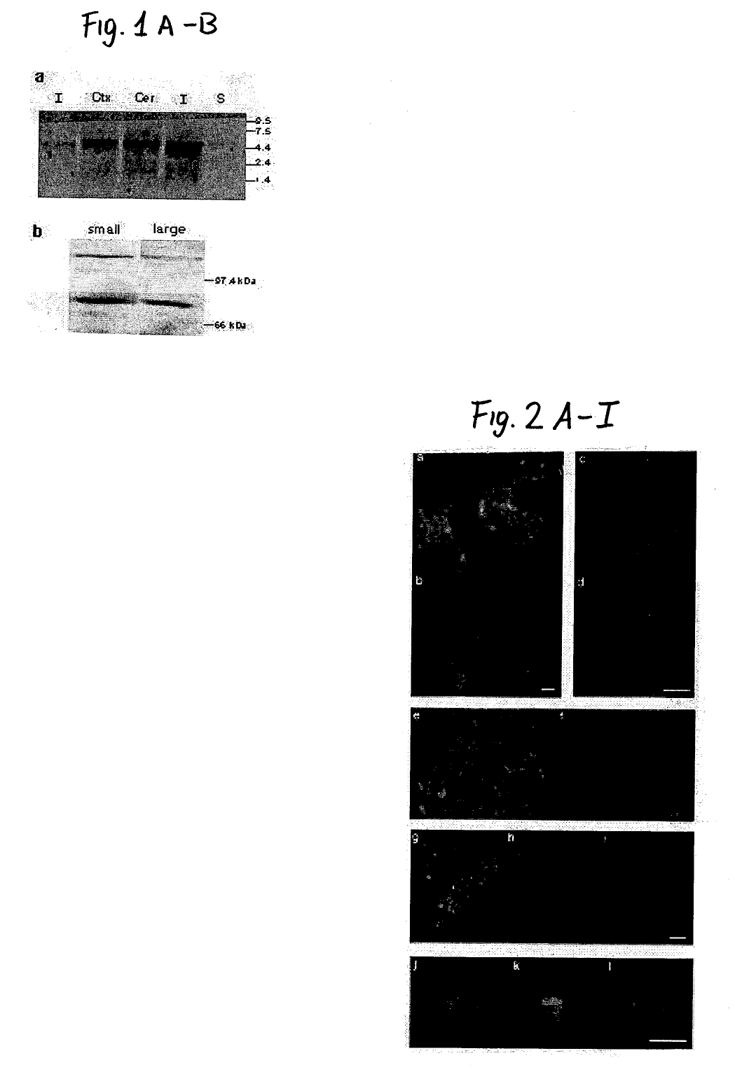

High-affinity binding sites for GABA were previously reported on cells within islets and over intralobular ducts²⁵, the site of islet neogenesis⁷⁻⁹. To investigate whether GABA_{B} receptors might be responsible for this binding, we employed immunocytochemical methods with two antibodies to the GABA_{B} receptor (synthesized against the N-terminus or the C-terminus of GBR1) and compared the localization of receptor to that of GABA, detected with an anti-GABA antibody. GBR1 immunoreactivity was found on islets where it co-localized with GABA (FIGS. 2A and B). Using the C-terminal antibody, prominent labeling for GBR1 was observed on all isolated islets smaller than 150 μm diameter (FIG. 2C). Within this group, 97% of cells expressed the GABA_{B} receptor and, of those, 78% also contained insulin (FIG. 2D). Thus, all insulin-positive cells in small islets demonstrated GABA_{B} receptor immunoreactivity. Significantly smaller amounts of GABA_{B} receptor protein were detected on large islets, however (FIG. 2E). The differential expression of GBR1 in small vs. large islets was confirmed by Western blot analysis (FIG. 1C).

Immunolabeling with the N-terminal antibody to GBR1 was associated both with the cytoplasm and with the plasma membrane (FIG. 2F), as would be predicted by the full length and truncated transcripts cloned from islet mRNA (FIG. 1). This labeling by the N-terminal antibody is likely to represent the presence of both the long membrane bound form of the GABA_{B} receptor and a truncated cytoplasmic form, which has been shown to be soluble and secreted²⁶ when expressed heterologously.

Immunocytochemistry was also employed to localize GBR2, the heterodimeric partner of GBR1^{20,22}. As with GBR1, GBR2 localized to small islets, but unlike GBR1, it appeared to selectively associate with the plasma membrane, with little or no labeling of the cytosol (FIG. 2G). Thus, it appears likely that at least some forms of GBR1 are expressed in islet cells in the absence of physical association with GBR2.

GABA and GABA_{B} Receptors Near to and on Ductal Epithelium

New islets are formed through the division and differentiation of cells in ductal epithelium⁷⁻⁹. As a consequence, small clusters of β cells in newly-developing islets can be observed near pancreatic ducts⁷. Such islet cells labeled positively for both the GABA_{B} receptor (GBR1 C terminus) and for GABA (FIGS. 3A,B). In addition, interlobular ducts exhibited GABA_{B} receptor immunoreactivity (confirming the earlier GABA receptor binding studies of Reusens et al.²⁵) as well as immunoreactive GABA, suggesting that the expression of these GABA signaling molecules precedes the appearance of the islets themselves (FIGS. 3C-E). This further implies the possibility that GABA plays some role in early events in islet neogenesis.

GABA_{B} Receptors Stimulate Islet Cell Growth

Islets contain cells capable of proliferation in response to growth factors^{9,11-13}. In the fetus and neonate, this compartment is as large as 10%, but even in adults 3% of islet cells are capable of division. Through a combination of proliferation in this compartment and growth of fully differentiated cells, islet mass can increase by 50% in just 96 hours of glucose infusion¹⁵. This increase in mass is selective for β cells.

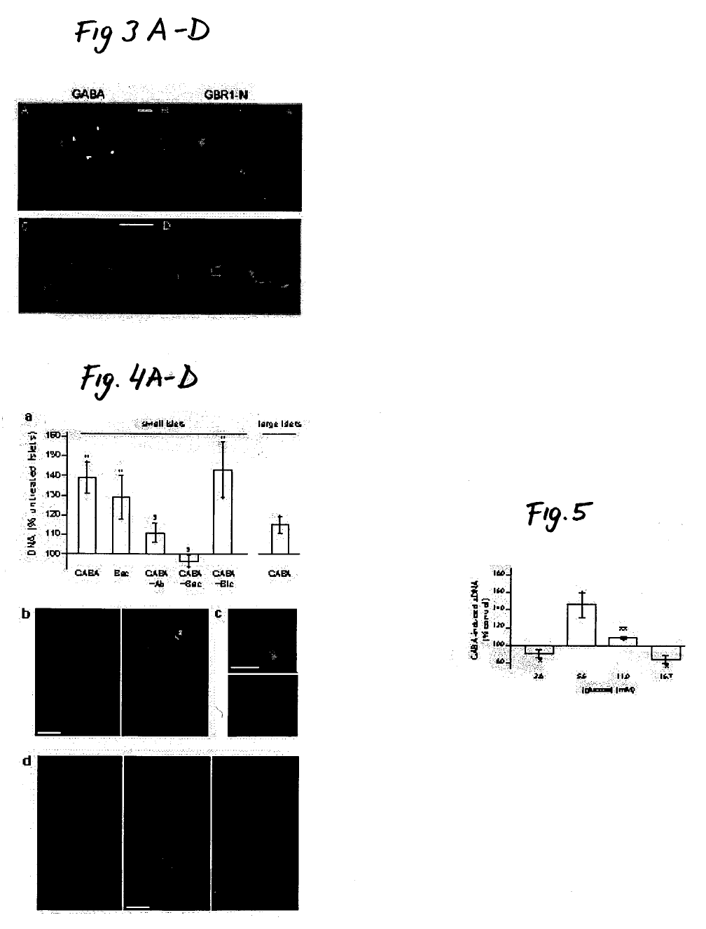

To examine the potential function of GABA_{B} receptor signaling in islet tissue, islets were enzymatically isolated from exocrine pancreas and studied in vitro. Islets were size-selected for these studies because of our observation that small islets (<150 μm diameter) express GABA_{B} receptors more abundantly. Islets (equivalent to 10,000 cells per well of a 96 well culture plate) were incubated for 45 minutes with (or without) 100 μM GABA, washed once with growth medium M199, maintained for 4 hours at 37° C., and subsequently analyzed for total DNA content using a Cyquant (Molecular Probes) fluorescence assay. Small islets treated with GABA exhibited a 45±*% increase in DNA synthesis in the 4.75 hours following the start of GABA exposure (FIG. 4A). Large islets, by contrast, were much less responsive to GABA; increasing only by 15±4.5% in the same time period. The growth-promoting effect of GABA was mimicked by the selective GABA_{B} receptor agonist, S-baclofen and inhibited with either 20H-saclofen (a selective inhibitor of GABA_{B} receptors) or the N-terminal antibody to the GABA_{B} receptor (FIG. 4A). The GABA_{A} receptor antagonist, bicuculline, was without effect.

[³H]thymidine incorporation was employed as an alternative assay for DNA synthesis, with similar results to those of the fluorescence assay. The labeled nucleotide was added to the culture medium and was present throughout the 4.75 hour experimental period. GABA, and the agonist baclofen, acting via GABA_{B} receptors produced a respective 143±19% and 115±4% increase in [³H]thymidine incorporation (FIG. 4B). Taken together, these results demonstrate that GABA exerts a mitogenic action on islets that is mediated through GABA_{B} receptors.

Labeling the Proliferative Compartment in Islets

To identify which cells in islets underlie the increase in DNA synthesis, cells treated with GABA were exposed to BrdU, to label the dividing cells, and compared to control cells. As has been reported previously^{17,8}, we found that the proliferative compartment within islets was small; in the 4.75 hours, only islets treated with GABA exhibited detectable BrdU incorporation (FIG. 4C). Tissue sections frequently showed islets that appeared to have formed around a lumen; in such sections (e.g. FIGS. 4D,E), BrdU labeling was abundant in cells juxtaposed to the lumenal layer. Given the brief incorporation period (4 hours following GABA exposure), such profiles imply that the responsive cells are likely to be synchronized in S phase. These results suggest that islets have the capacity to respond rapidly when exposed to GABA and that the most responsive cells are those near ductal epithelium.

GABA_{B} Receptors Mediate Glucose-Stimulated Mitogenesis

Others have shown that islet mass can increase by as much as 50% in just 96 hours of glucose infusion in vivo, an effect that results from increases in both cell size and cell number¹⁵. Given that metabolism of glucose alone promotes secretion from β cells, we tested the hypothesis that glucose-stimulated proliferation in islets involves the activation of GABA_{B} receptors by GABA released during glucose application. Islets were exposed to 5.6 mM glucose (a maximal proliferative stimulus in human islets) for 4.75 hours in the presence or absence of 20H-saclofen or the GBR1 N-terminal antibody. Glucose promoted a small, but significant, 7±1% stimulation of control islets during this time (FIG. 5A). Islets exposed to 20H-saclofen, by contrast, exhibited a statistically-significant reduction in DNA (92+4% of DNA content measured at the beginning of the incubation period) (FIG. 5B); islets exposed to the GBRI1 N-terminal antibody exhibited a similar reduction in DNA synthesis (90±5% of control). These results support the idea that GABA_{B} receptors mediate the proliferative actions of glucose and further imply that tonic GABA_{B} receptor signaling is required for islet maintenance.

To further evaluate the interactions between glucose and GABA_{B} receptor activation, we measured GABA-mediated proliferation over a range of glucose concentrations. DNA synthesis stimulated by 45 min exposure to 100 μM GABA peaked at moderate glucose concentrations (5.6 mM, FIG. 5C), whereas higher concentrations of glucose (16.7 mM) suppressed the stimulatory effect of GABA.

It is to be understood that while the invention has been described in conjunction with the preferred specific embodiments thereof that the foregoing description as well as the examples that follow are intended to illustrate and not limit the scope of the invention. Other aspects, advantages and modifications within the scope of the invention will be apparent to those skilled in the art to which the invention pertains.

### Expression of GABA_{B}-Like Receptors

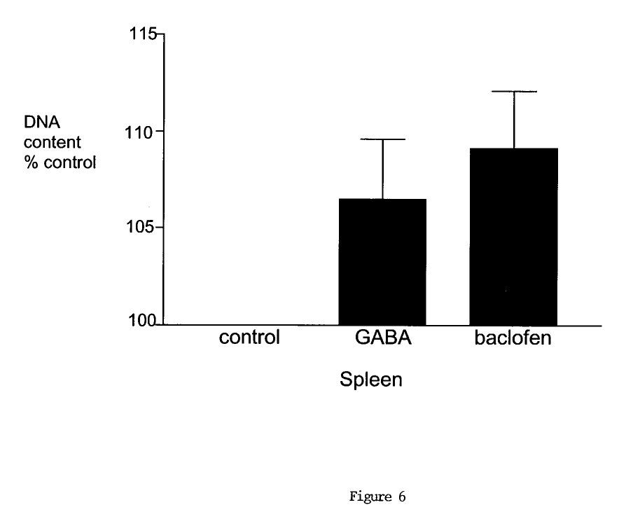

An experiment to test the ability of GABA and the GABA_{B} receptor agonist to stimulate growth of other tissues was undertaken. Rat spleen was mechanically dissociated in trypsin, washed, and plated identical to the experiments using rat islets, described above. The dissociated tissue was cultured in the presence of 100 micromolar GABA or baclofen for 45 minutes and compared to cultured but untreated dissociated spleen. After 45 minutes the media was changed and the tissue incubated in fresh medium for four hours at 37°. DNA measurements were made by the Cyquant assay by Molecular Probes. The results of this experiment are shown in FIG. 6, which demonstrates that the presence of GABA and the GABA receptor agonist baclofen promoted an increase in the DNA content of cultured spleen cells when compared to untreated spleen cells. These results indicate that GABA and GABA receptor agonists promote proliferation of spleen cells in culture.

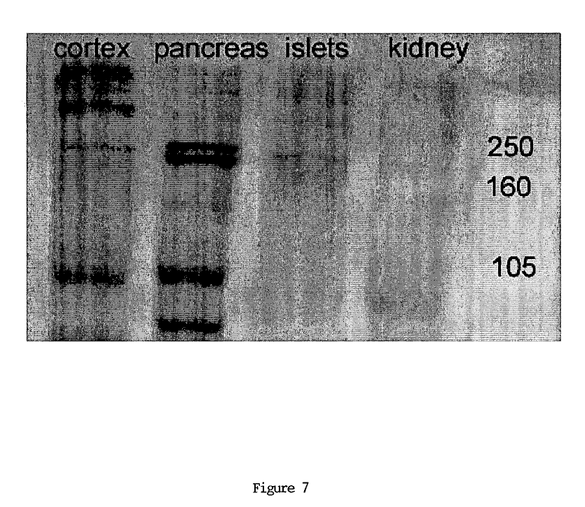

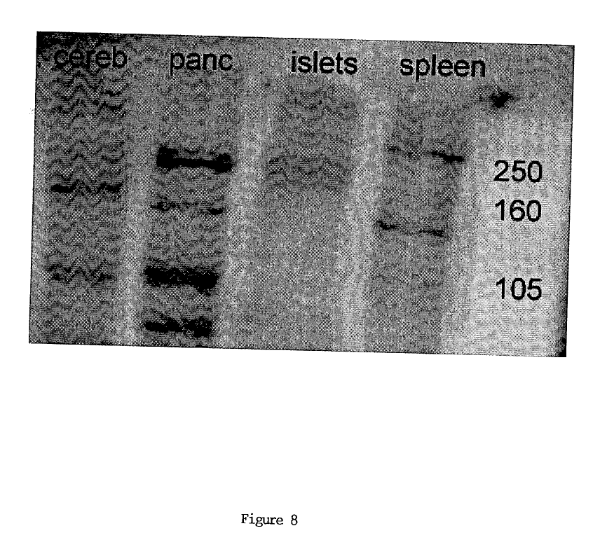

The presence of GABA_{B}-like receptors can be seen in a variety of tissues by Western blotting analysis using a GABA_{B} receptor antibody (FIGS. 7 and 8). By immunoblot, multiple isoforms of the GABA_{B} receptor are labeled in each tissue studied, including those meant to be a negative control. The antibody used was raised to a peptide in the ligand binding domain of the GABA_{B} receptor consisting of the following residues: RRDILPDYELKLIHHD. There are prominent doublets in pancreas and islets that are 160 kD in size. These bands are labeled less prominently in cerebellum. The most abundant isoform in spleen is 260 kD and may represent a dimer, or a much larger receptor. A protein of 130 kD is also labeled in pancreas and spleen, which is the size of the GABA_{B} receptor isoform originally cloned from brain by Kaupmann et al in 1997. Cerebellum, pancreas and spleen also express a protein labeled by this antibody of 103 kD in size, which was also seen in immunoblots of the original GABA_{B} receptor clones by Kaupmann, et al. In our second immunoblot, proteins are labeled in the kidney lysate of identical size to those labeled in spleen.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

### REFERENCES

The references cited below and incorporated throughout the application are incorporated herein by reference.

1. Kahn, C. R., Vincent,D., and Doria, A. Genetics of Type II diabetes Annu. Rev. Med. 47 509-531 (1996).

2. Kahn, B. B. Type 2 diabetes: when insulin secretion fails to compensate for insulin resistance. Cell 92 593-596. (1998).

3. Michalik, M. & Erecinska, M. GABA in pancreatic islets: metabolism and function. Biochem. Pharm. 44,1-9 (1992).

4. Rorsman, P., Berggren, P-O., Bokvist, K., Ericson, H., Mohler, H., Ostenson, C-G. and Smith, P. A., Glucose-inhibition of glucagon secretion involves activation of GABA_{A}receptor chloride channels. Nature 341 233-236 (1989).

5. Smismans, A., Schuit, F., Pipeleers, D., Nutrient regulation of gamma-aminobutyric acid release from islet beta cells. Diabetologia 40 1411-1415 (1997).

6. White, J. H., Mclllhinney R. A. J., Wise, A., Ciruela, F., Chan, W-Y., Emson, P. C., Billinton, A., and Marshall, F. H. The GABA_{B} receptor interacts directly with the related transcription factors CREB2 and ATFx. Proc Nat Acad Sci 97 13967-13972 (2000).

7. Bouwens, L., and Pipeleers, D.G. Extra-insular beta cells associated with ductules are frequent in adult human pancreas. Diabetologia 41, 629-633 (1998).

8. Slack, J. M. W., Developmental biology of the pancreas. Development 121 1569-1580 (1995).

9. Nielsen, J. H., Galsgaard, E. D., Moldrup, A., Friedrichsen, B. N., Billestrup, N., Hansen, J. A., Carlsson, C. Regulation of β-cell mass by hormones and growth factors. Diabetes 50 Supplement 1 S25-S29. (2001).

10. Taylor, S. I. Deconstructing type 2 diabetes. Cell 97 9-12

11. Hellerström, C., Sjöholm, Å., Swenne, I. Effects of growth hormone and related growth factors on DNA replication and insulin production in pancreatic islet β-cells. Acta Paediatr. Scand. [Suppl] 377, 55-62 (1991).

12. Hart, A. W., Baeza, N., Apelqvist, A. & Edlund, H., Attenuation of FGF signaling in mouse □-cells leads to diabetes. Nature 408, 864-868 (2000).

13. Brelje, T. C., & Sorenson, R. L. Role of Prolactin versus growth hormone on islet β-cell proliferation in vitro: Implications for pregnancy. Endocrinology 128, 45-57 (1991)

14. Xu, G., Stoffers, D. A., Habener, J. F., Bonner-Weir, S. Extendin-4 stimulates both β-cell replication and neogenesis, resulting in increased □-cell mass and improved glucose tolerance in diabetic rats. Diabetes 48 2270-2276 (1999).

15. Bonner-Weir, S., Deery, D., Leahy, J. L., and Weir, G. C., Compensatory growth of pancreatic β-cells in adult rats after short-term glucose infusion. Diabetes 38 49-53 (1989).

16. Swenne, I. Pancreatic beta-cell growth and diabetes mellitus. Diabetologia. 35, 193-201 (1992).

17. Buteau, J., Roduit, R., Susini, S., Prentki, M., Glugagon-like peptide-1 promotes DNA synthesis, activates phosphatidylinositol 3-kinase and increases transcription factor pancreatic and duodenal homeobox gene 1 (PDX-1) DNA binding activitly in β (INS-1)-cells. Diabetologia 42 856-864. (1999).

18. Reetz, A., Solimena, M., Matteoli, M., Folli, F., Takei, K., and De Camilli, P. GABA and pancreatic β-cells: colocalization of glutamic acid decarboxylase (GAD) and GABA with synaptic-like microvesicles suggests their role in GABA storage and secretion. EMBO 10, 1275-1284 (1991).

19. Betz, H., Ligand-gated ion channels in the brain: The amino acid receptor superfamily. Neuron 5 383-392 (1990).

20. Bowery, N. G., and Enna, S. J. β-aminobutyric acid B receptors: first of the functional metabotropic heterodimers. J. Pharmacol. and Exper. Ther. 292, 2-7 (1999).

21. Kaupmann, K., Huggel, K., Heid, J., Flor, P. J., Bischoff, S., Mickel, S. J., McMaster, G., Angst, C., Bittiger, H., Froestl, W., and Bettler, B. Expression cloning of GABA_{B} receptors uncovers similarity to metabotropic glutamate receptors. Nature 386, 239-246 (1997).

22. Kaupmann, K. et al. GABA_{B}-receptor subtypes assemble into functional heteromeric complexes. Nature 396, 683-687 (1998).

23. Liang G., & Hai, T., Characterization of Human Activating Transcription factor 4, a transcriptional activator that interacts with multiple domains of cAMP-responsive element binding protein (CREB)-binding protein (CBP). J. Biol. Chem. 272, 24088-24095 (1997).

24. Schwarz D. A., Barry G., Eliasof S. D., Petroski R. E., Conlon P. J., Maki R. A. Characterization of gamma-aminobutyric acid receptor GABA_{B}(1 e), a GABA_{B}(1) splice variant encoding a truncated receptor.J Biol Chem 275 32174-32181 (2000).

25. Reusens-Billen, B. Pirlot,X., Remacle, C., Hoet, J. J., and de Gasparo, M., Localization of GABA high-affinity binding sites in the pancreas of neonatal rat Cell and Tissue Res 235 503-508. (1984).

26. 2. Gerber I I I, J. C. & Hare, T. A. Gamma-aminobutyric acid in peripheral tissue, with emphasis on the endocrine pancreas. Diabetes. 28, 1073-1076 (1979).

27. Poitout, V., & Robertson, R. P., An integrated view of β-cell dysfunction in type II diabetes. Annu. Rev. Med. 47 69-83 (1999).

28. Kash, s. F., Condie, B. G., Baekkeskov, S., Glutamate decarboxylase and GABA in pancreatic islets: lessons from knock-out mice. Horm Metab Res 31 340-344. (1999).

29. Beales et al., Acta Diabetol. 32(1):53-6 (1995).

30. Bridgett et al., Diabetes 47(12):1848-56. (1998).

31. Castagliuolo, I., Valenick, L., Liu, J., and Pothoulakis, C., Epidermal growth factor receptor transactivation mediates substance P-induced mitogenic responses in U-373 MG cells. J. Biol. Chem 275 26545-26550. (2000).

32. Lee, S-L., Wang, W-W., Finlay, G. A., and Fanburg, B. L. Serotonin stimulates mitogenactivated protein kinase activity through the formation of superoxide anion. Am. J. Physiol. 277 L282-L291(1999).

I claim:

1. A method for stimulating tissue growth, comprising administering to a mammal an effective amount of a compound selected from the group consisting of GABA, a GABA_{B} receptor agonist, a GABA_{B}-like receptor agonist, and a pharmaceutically acceptable salt thereof.

2. The method of claim 1, wherein the tissue growth is stimulated in a subject in need thereof.

3. The method of claim 1, wherein the compound is selected from the group consisting of:
4-aminobutanoic acid (GABA),
4-amino-3-(4-chlorophenyl)butanoic acid (baclofen),
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
(3-aminopropyl)phosphonous acid,
(4-aminobut-2-yl)phosphonous acid, sodium butyrate,
(3-amino-2-methylpropyl)phosphonous acid,
(3-aminobutyl)phosphonous acid,
(3-amino-2-(4-chlorophenyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonous acid,
[3-amino-2-(4-methoxyphenyl)propyl]phosphonous acid,
[3-amino-2-(4-chlorophenyl)-2-hydroxypropyl]phosphonous acid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methylphosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid, and
3-aminopropylsulfinic acid.

4. A method for regenerating tissue, comprising selecting a host in need of tissue regeneration, and administering to said host an effective amount of a compound selected from the group consisting of GABA, a GABA_{B} receptor agonist, a GABA_{B}-like receptor agonist, and a pharmaceutically acceptable salt thereof.

5. The method of claim 4, wherein the compound is selected from the group consisting of:
4-aminobutanoic acid (GABA),
4-amino-3-(4-chlorophenyl)butanoic acid (baclofen),
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
(3-aminopropyl)phosphonous acid,
(4-aminobut-2-yl)phosphonous acid,
sodium butyrate,
(3-amino-2-methylpropyl)phosphonous acid,
(3-aminobutyl)phosphonous acid,
(3-amino-2-(4-chlorophenyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonous acid,
[3-amino-2-(4-methoxyphenyl)propyl]phosphonous acid,
[3-amino-2-(4-chlorophenyl)-2-hydroxypropyl]phosphonous acid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methylphosphinic acid,
(3-aminopropyl)hydroxymethylphosphinic acid,
(5-aminopent-3-yl)methylphosphinic acid,
(4-amino-1, 1,1-trifluorobut-2-yl)methylphosphinic acid,
(3-amino-2-(4-chlorophenyl)propyl)sulfinic acid, and 3-aminopropylsulfinic acid.

6. A method for stimulating islet growth, comprising administering to a mammal an effective amount of a compound selected from the group consisting of GABA, a GABA_{B} receptor agonist, a GABA_{B}-like receptor agonist, and a pharmaceutically acceptable salt thereof.

7. The method of claim 6, wherein the GABA_{B} receptor agonist is selected from the group consisting of:
4-aminobutanoic acid (GABA),
4-amino-3-(4-chlorophenyl)butanoic acid (baclofen),
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
(3-aminopropyl)phosphonous acid,
(4-aminobut-2-yl)phosphonous acid,
sodium butyrate,
(3-amino-2-methylpropyl)phosphonous acid,
(3-aminobutyl)phosphonous acid,
(3-amino-2-(4-chlorophenyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonous acid,
[3-amino-2-(4-methoxyphenyl)propyl]phosphonous acid,
[3-amino-2-(4-chlorophenyl)-2-hydroxypropyl]phosphonous acid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methylphosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid, and
3-aminopropylsulfinic acid.

8. A method for stimulating islet growth, comprising administering to islets in culture an effective amount of a compound selected from the group consisting of GABA, a GABA_{B} receptor agonist, a GABA_{B}-like receptor agonist, and a pharmaceutically acceptable salt thereof.

9. The method of claim 8, wherein the GABA_{B} receptor agonist is selected from the group consisting of:
4-aminobutanoic acid (GABA),
4-amino-3-(4-chlorophenyl)butanoic acid (baclofen),
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
(3-aminopropyl)phosphonous acid,
(4-aminobut-2-yl)phosphonous acid, sodium butyrate,
(3-amino-2-methylpropyl)phosphonous acid,
(3-aminobutyl)phosphonous acid,
(3-amino-2-(4-chlorophenyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonous acid,
[3-amino-2-(4-methoxyphenyl)propyl]phosphonous acid,
[3-amino-2-(4-chlorophenyl)-2-hydroxypropyl]phosphonous acid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methylphosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid, and
3-aminopropylsulfinic acid.

10. A method for treatment of diabetes mellitus, comprising administering to a mammal in need of such treatment an effective amount of a compound selected from the group consisting of GABA, a GABA_{B} receptor agonist, a GABA_{B}-like receptor agonist, and a pharmaceutically acceptable salt thereof.

11. The method of claim 10, wherein the diabetes mellitus is insulin dependent diabetes mellitus.

12. The method of claim 10, wherein the diabetes mellitus is non-insulin dependent diabetes mellitus.

13. The method of claim 10, wherein the GABA_{B} receptor agonist is selected from the group consisting of:
4-aminobutanoic acid (GABA),
4-amino-3-(4-chlorophenyl)butanoic acid (baclofen),
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
(3-aminopropyl)phosphonous acid,
(4-aminobut-2-yl)phosphonous acid, sodium butyrate,
(3-amino-2-methylpropyl)phosphonous acid,
(3-aminobutyl)phosphonous acid,
(3-amino-2-(4-chlorophcnyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonous acid,
[3-amino-2-(4-methoxyphenyl)propyl]phosphonous acid,
[3-amino-2-(4-chlorophenyl)-2-hydroxypropyl]phosphonous acid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)mcthylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)mcthylphosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid, and
3-aminopropylsulfinic acid.
